(19) United States
(12) Patent Application Publication
(10) Pub. No.: US 2021/0154422 A1
(43) Pub. Date: May 27, 2021

(54) CPAP MANAGEMENT SYSTEM AND MANAGEMENT METHOD OF MANAGING A PLURALITY OF CPAP DEVICES

(71) Applicant: CHEST M.I., INCORPORATED, Tokyo (JP)

(72) Inventor: Satoru Hisahara, Tokyo (JP)

(21) Appl. No.: 17/044,862

(22) PCT Filed: Apr. 4, 2019
(86) PCT No.: PCT/JP2019/015031

§ 371 Date: Oct. 1, 2020

(30) Foreign Application Priority Data
Apr. 5, 2018 (JP) 2018-073262

(51) Int. Cl. A61M 16/00; G16H 40/67; G16H 50/20; G16H 20/40; G16H 40/40; G16H 50/70

(52) U.S. Cl. CPC A61M 16/024; G16H 40/67; G16H 50/20; A61M 2205/52; G16H 40/40; G16H 50/70; G16H 20/40

(57) ABSTRACT

The CPAP management system includes a data processor configured to process data on a subject transmitted from a CPAP device and an analytical predictor configured to extract data for a second period until a date on which use of the CPAP device is stopped, from data on a subject whose period of non-use of the CPAP device is a first period or longer included in data on a plurality of subjects stored in a server that stores therein the data on the subjects transmitted from a plurality of the CPAP devices, and output a prediction result as to whether the subject potentially becomes a dropout from the therapeutic device in the future, based on the data for the second period.

### FIELD

The present disclosure relates to a CPAP management system and a management method of managing a plurality of CPAP devices.

### BACKGROUND

The recent growing awareness of quality of life (QOL) has led to the widespread use of oxygen therapy and medical ventilation at home. Respiratory therapy for patients with sleep apnea syndrome (SAS) has been commonly known.

For therapy for sleep apnea syndrome, continuous positive airway pressure (CPAP) devices have been used, which forcedly deliver the air into the airway with a fan, with a mask fixed to the patient's face. The CPAP device has a structure including: a main body device that contains a fan and a controller and is placed away from a human body; and a hose that connects the main body device to a mask fixed to the face and through which the air is delivered.

For example, Patent Literature 1 discloses a CPAP device that always maintains therapeutic pressure to the optimum level relative to the patient's airway resistance.

### CITATION LIST

### Patent Literature

Patent Literature 1: Japanese Patent Application Laid-open No. 2008-264181

### SUMMARY

### Technical Problem

Unfortunately, almost 30% of patients drop out of therapy using CPAP devices at an early stage (for example, half a year) after starting the therapy using CPAP devices. Possible reasons for the dropping out include aversion to wearing the device while sleeping and lack of appropriate prescriptions (settings of the therapeutic devices). It is therefore important to provide appropriate follow-ups so that patients will not drop out from the therapy using the CPAP devices.

The present disclosure is made in view of the problem above and aims to provide a CPAP management system capable of predicting whether a subject potentially becomes a dropout from a therapy device in the future, and a management method of managing a plurality of CPAP devices.

### Solution to Problem

According to an aspect, a CPAP management system includes: a data processor configured to process data on a subject transmitted from a CPAP device; and an analytical predictor configured to extract data for a second period until a date on which use of the CPAP device is stopped, from data on a subject whose period of non-use of the CPAP device is a first period or longer included in data on a plurality of subjects stored in a server that stores therein the data on the subjects transmitted from a plurality of the CPAP devices, and output a prediction result as to whether the subject potentially becomes a dropout from the CPAP device in the future, based on the data for the second period.

According to another aspect, a management method of managing a plurality of CPAP devices includes: a first step of storing data on a plurality of subjects transmitted from the CPAP devices into a server, extracting, from data on a subject whose period of non-use of a corresponding CPAP device of the CPAP devices is equal to or longer than a first period included in the data on subjects stored in the server, data for a second period until a date on which use of the CPAP device is stopped, and creating reference data about a tendency of a dropout from the CPAP devices; a second step of pre-processing data on a subject transmitted from a CPAP device of the CPAP devices; and a third step of analyzing the data on the subject acquired in the second step based on the reference data created in the first step and outputting a warning that the subject potentially drops out of therapy with the CPAP device.

### Advantageous Effects of Invention

According to the present disclosure, whether a subject potentially becomes a dropout from the CPAP device in the future can be predicted.

### BRIEF DESCRIPTION OF DRAWINGS

FIG. 1 is a diagram illustrating a configuration of a CPAP management system in a first embodiment.

FIG. 2 is a diagram illustrating a configuration of a data analysis and prediction apparatus in the first embodiment.

FIG. 3 is a diagram illustrating an example of data on a subject stored in a server.

FIG. 4 is a diagram illustrating an example of data on a subject stored in a server.

FIG. 5 is a diagram illustrating an example of data suspicious for dropping out of therapy with a CPAP device but is not handled as data on dropping out for a fair reason.

FIG. 6 is a diagram illustrating an example of data suspicious for dropping out of the therapy with a CPAP device.

FIG. 7 is a diagram illustrating an example of use information on the use of a CPAP device created for each subject.

FIG. 8 is a flowchart illustrating a procedure of monitoring a sign of dropping out of a CPAP device.

FIG. 9 is a diagram illustrating another configuration of the CPAP management system.

FIG. 10 is a flowchart illustrating a procedure of preventing dropping out of a CPAP device.

FIG. 11 is a diagram illustrating a display example of mask information displayed on an information terminal.

FIG. 12 is a diagram illustrating a configuration of the CPAP management system in a second embodiment.

FIG. 13 is a diagram illustrating a database for determining a sign of dropping out of a CPAP device.

FIG. 14 is a flowchart illustrating a procedure of monitoring a sign of dropping out of a CPAP device.

### DESCRIPTION OF EMBODIMENTS

Modes for carrying out the present invention (embodiments) will be described in detail with reference to the drawings. It should be noted that the present invention is not limited by the description of the embodiments below. The components described below include those easily conceived by a person skilled in the art and those substantially identical. The components of the embodiments described below can be combined as appropriate. Hereinafter, a CPAP device will be described as an example of a therapeutic device. CPAP refers to continuous positive airway pressure therapy.

### First Embodiment

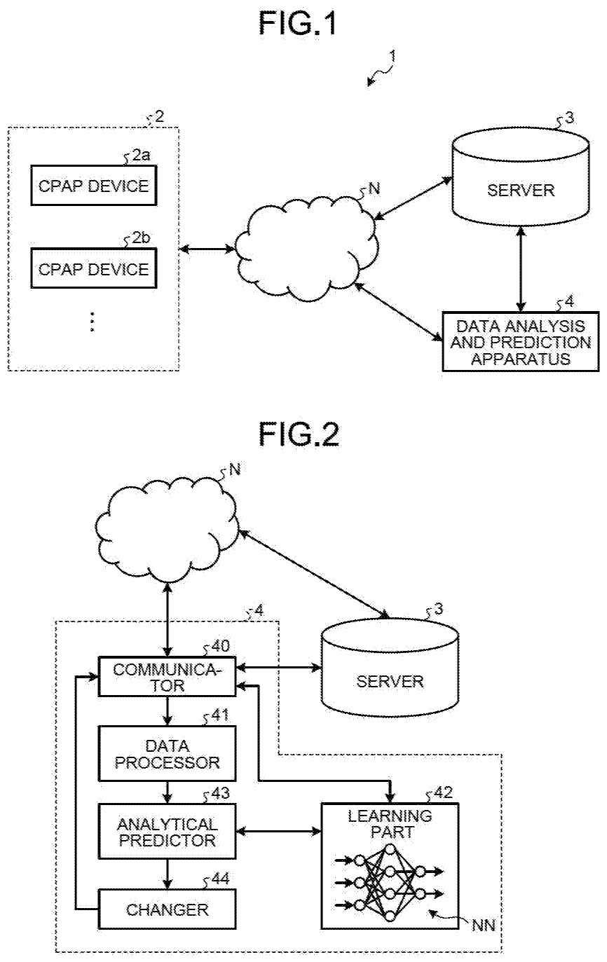

FIG. 1 is a diagram illustrating a configuration of a CPAP management system 1 in a first embodiment. The CPAP management system 1 includes a plurality of CPAP devices 2a, 2b, . . . , a server 3, and a data analysis and prediction apparatus 4, which are connected through a network N. The CPAP management system is an information processing system. Hereinafter a plurality of CPAP devices 2a, 2b, . . . may each be called a “CPAP device 2”. The data analysis and prediction apparatus 4 analyzes data for each of the CPAP devices 2a, 2b, . . . and predicts individually whether the subjects of the CPAP devices 2a, 2b, potentially become dropouts in the future.

CPAP refers to a therapy that delivers the air pressurized by the CPAP device 2 to the airway through the nose and expands the airway to prevent apnea during sleep. The CPAP device 2 includes a tube for feeding the air at a preset pressure and a mask applied to the nose.

The settings of the CPAP device 2, such as the magnitude of pressure, are made by a doctor in accordance with a medical state of the subject. Many researches have proven the efficacy of CPAP for sleep apnea syndrome: for example, subjects who have received CPAP therapy live longer life when CPAP therapy is conducted, compared with when CPAP therapy is not conducted. CPAP therapy is now widely used as a standard therapy for patients with sleep apnea syndrome (SAS).

The CPAP device 2 transmits information that provides the use state of the device, such as information about the days of use of the device, to the server 3 through the network N, at a predetermined timing.

The server 3 receives and stores the information transmitted from the CPAP device 2. More specifically, data on a plurality of subjects transmitted from a plurality of CPAP devices 2a, 2b, . . . are stored in the server 3. The server 3 is a computer and includes a central processing unit (CPU), a random access memory (RAM), a read only memory (ROM), and an internal storage device such as a hard disc drive (HDD). The server 3 may be called a cloud server.

The data analysis and prediction apparatus 4 analyzes the information stored in the server 3 that is transmitted from the CPAP device 2 and predicts whether the subject potentially becomes a dropout from the CPAP device 2 in the future. A specific configuration and operation of the data analysis and prediction apparatus 4 is described below.

FIG. 2 is a diagram illustrating a configuration of the data analysis and prediction apparatus 4 in the second embodiment. The data analysis and prediction apparatus 4 is a computer and includes a central processing unit (CPU), a random access memory (RAM), a read only memory (ROM), and an internal storage device such as a hard disc drive (HDD). The data analysis and prediction apparatus 4 may include a graphics processing unit (GPU), and the CPU may perform computation. With the hardware in combination with execution of the software, the data analysis and prediction apparatus 4 includes a communicator 40, a data processor 41, a learning part 42, and an analytical predictor 43.

The communicator 40 performs communication with the server 3 and the CPAP devices 2. The data processor 41 processes data on a subject transmitted from a CPAP device 2 through the communicator 40. In the present embodiment, the data processor 41 reads data on a subject stored in the server 3 through the communicator 40 and processes the read data on a subject. However, the present invention is not limited to this configuration. The data processor 41 may directly receive and process data on a subject transmitted from a CPAP device 2 through the communicator 40.

For example, the data processor 41 performs pre-processing on the data on a subject. The pre-processing refers to a process of extracting necessary data from the data on a subject or a process of processing the data into a format suitable for the processing by the analytical predictor 43.

The learning part 42 performs communication with the server 3 through the communicator 40. The learning part 42 learns based on data on a dropout who drops out of therapy using the CPAP device 2 and generates a neural network NN based on the data on subjects stored in the server 3. Learning in the present embodiment may be supervised learning or may be unsupervised learning.

Data on a dropout who drops out of therapy using the CPAP device 2 will now be described. In the present embodiment, it is assumed that the use time of the CPAP device 2 is managed in chronological order for each subject.

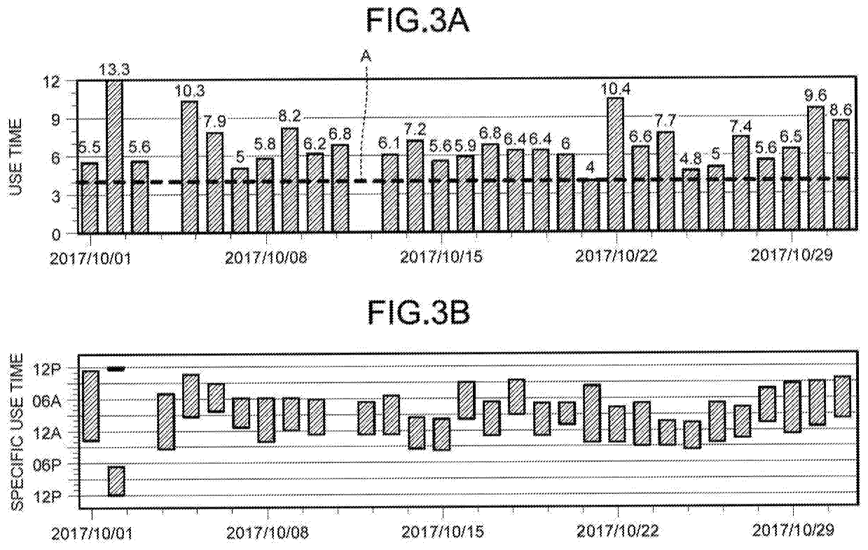

FIG. 3 illustrates an example of data on a subject stored in the server 3, which is an example of data on a subject who keeps having therapy using the CPAP device 2. “A” in FIG. 3(a) represents a certain length of time (four hours). FIG. 3(a) illustrates an example of the daily use time of the CPAP device 2 for one month. FIG. 3(b) illustrates an example of specific time periods in which the CPAP device 2 is used.

It is understood that when appropriately used, the CPAP device 2 is used for a certain length of time or more in relatively regular time periods. The certain length of time is four hours in the example illustrated in FIGS. 3(a) and 3(b), but is not limited to four hours.

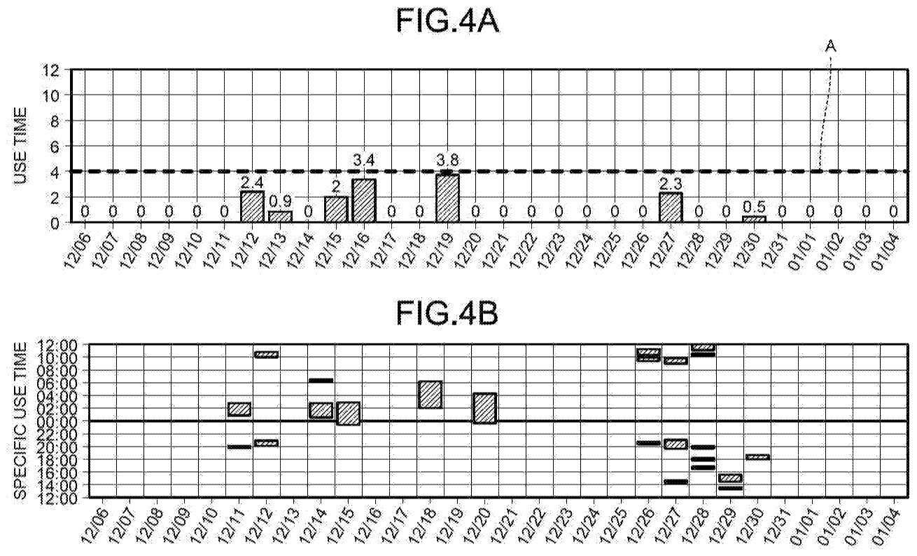

FIG. 4 illustrates an example of data on a subject stored in the server 3, which is an example of data on a subject who is not appropriately using the CPAP device 2. FIG. 4(a) illustrates an example of the daily use time of the CPAP device 2 for one month. “A” in FIG. 4(a) represents the certain length of time (four hours). FIG. 4(b) illustrates an example of specific time periods in which the CPAP device 2 is used.

It is understood that when the CPAP device 2 is not appropriately used, the irregular use of the CPAP device 2 is noticeable, and the use for the certain length of time or more is less frequent.

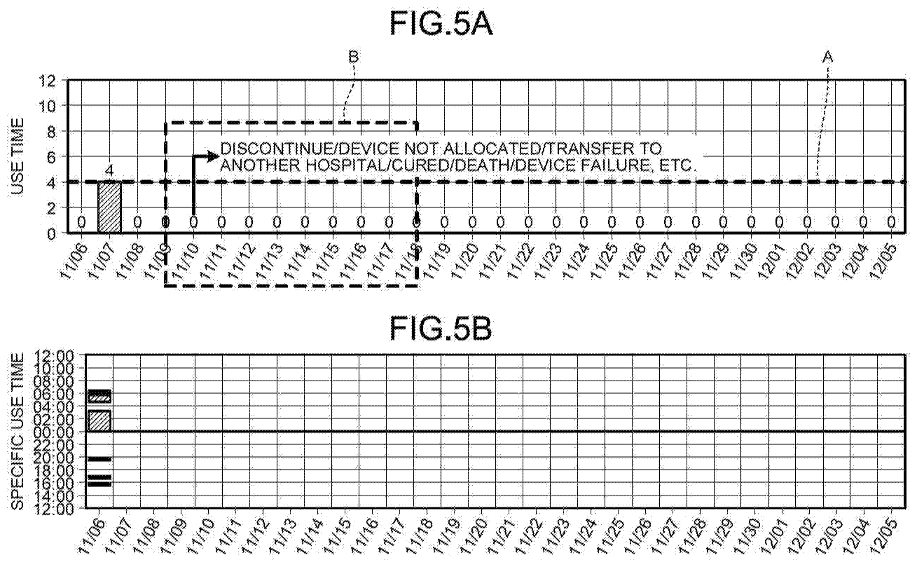

FIG. 5 is a diagram illustrating an example of data suspicious for dropping out of therapy with the CPAP device 2 but is not handled as data on dropping out for a fair reason. FIG. 5(a) illustrates an example of the daily use time of the CPAP device 2 for one month. FIG. 5(b) illustrates an example of specific time periods in which the CPAP device 2 is used.

In the period denoted by “B” in FIG. 5, data on the use time is not recorded. Possible reasons why there exists no data on the use time in FIG. 5 are: the subject is allowed to discontinue using the CPAP device 2; no CPAP device 2 is allocated; the subject is transferred to another hospital; the subject is cured; the subject dies; and the device fails. The data illustrated in FIG. 5 is not handled as data on dropping out, since the CPAP device 2 is not used for a fair reason. In other words, if the use status prior to the period denoted by B in FIG. 5 is appropriate as illustrated in FIG. 3, it is determined that there is a fair reason, and it is not determined as dropping out of the therapy with the CPAP device 2.

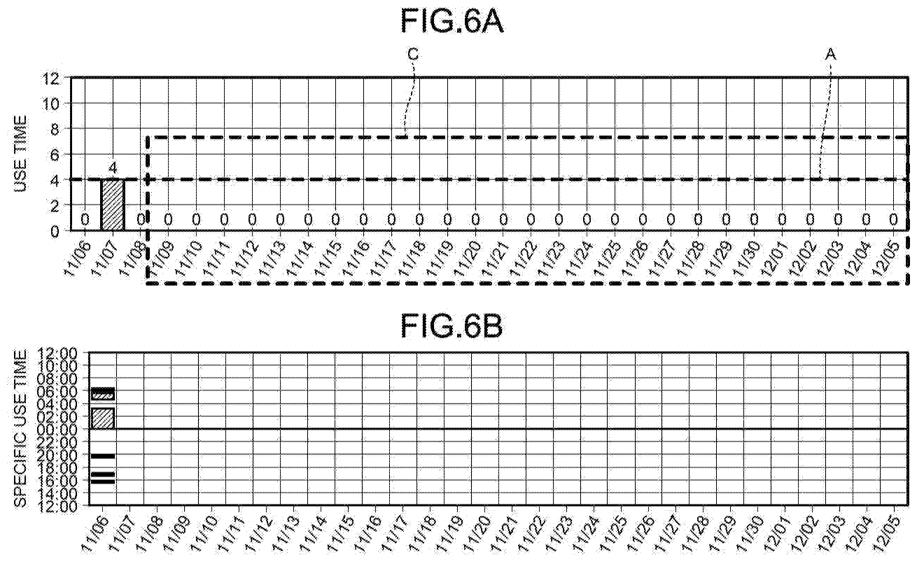

FIG. 6 is a diagram illustrating an example of data suspicious for dropping out of the therapy with the CPAP device 2. FIG. 6(a) illustrates an example of the daily use time of the CPAP device 2 for one month. FIG. 6(b) illustrates an example of specific time periods in which the CPAP device 2 is used.

FIG. 6 illustrates data in a case where the CPAP device 2 is not used for a certain period. The certain period is 14 consecutive days in the example illustrated in FIG. 6, but is not limited to 14 consecutive days.

When the use of the CPAP device 2 is resumed after non-use thereof for such reasons as traveling abroad and a failure of the CPAP device 2, it is not determined as dropping out of the therapy with the CPAP device 2. In the initial use of the CPAP device 2, the data often exhibits a pattern as illustrated in FIG. 6. The initial use refers to, for example, the use for less than half a year but is not limited to less than half a year.

The learning part 42 extracts data for a certain period on a subject suspected to drop out of the therapy with the CPAP device 2, as illustrated in FIG. 4, from the data on subjects stored in the server 3. The certain period is a second period, which will be described later. The learning part 42 learns based on the extracted data for the second period and generates a neural network NN related to a tendency of a dropout from the CPAP device 2 (the characteristics of the data suspicious for dropping out of the therapy with the CPAP device 2) from the result of learning.

The analytical predictor 43 utilizes the neural network NN generated by the learning part 42 to analyze data processed by the data processor 41 and predicts whether the subject potentially becomes a dropout from the CPAP device 2 in the future, based on the analysis result.

The data analysis and prediction apparatus 4 thus can predict whether a subject will drop out of the therapy with the CPAP device 2 in the future, by utilizing artificial intelligence (AI). For example, the result of prediction by the data analysis and prediction apparatus 4 may be presented to a professional, so that the professional can perform early and appropriate follow-ups for the subject and prevent the subject from dropping out of the therapy with the CPAP device 2. Examples of the professional include health workers such as doctors, laboratory technicians, and nurses.

Based on the data on a plurality of subjects, the learning part 42 may specify data on a subject whose period of non-use of the CPAP device 2 is a first period, and generate a neural network NN related to a tendency of a dropout from the CPAP device 2 from the result of learning based on the specified data on the subject.

The first period defines a period of non-use of the CPAP device 2 and is, for example, 14 days. Upon the lapse of the first period after the use of the CPAP device 2 is stopped, dropping out of the therapy with the CPAP device 2 is suspected. The data analysis and prediction apparatus 4 learns based on the data on each subject who has dropped out of the therapy with the CPAP device 2 and generates the neural network NN related to the tendency of the dropouts from the CPAP devices 2 from the result of learning.

The data analysis and prediction apparatus 4 therefore, by utilizing the generated neural network NN, can predict whether a subject potentially becomes a dropout from the CPAP device 2 in the future, depending on whether the data on the subject exhibits the same tendency as the subjects who have dropped out of the therapy with their CPAP devices 2.

For example, a professional can perform early and appropriate follow-ups for a subject likely to drop out of the therapy with the CPAP device 2, based on the prediction result of the data analysis and prediction apparatus 4, and can prevent the subject from dropping out of the therapy with the CPAP device 2.

The learning part 42 may extract data for the second period from the data on the specified subject, and generate a neural network NN related to a tendency of a dropout from the CPAP device 2 from the result of learning based on the extracted data for the second period.

The second period defines a period of extracting data from data on a subject and, for example, is six months until the first date on which the use of the CPAP device 2 is stopped. The learning part 42 generates the neural network NN related to the tendency of the dropout from the CPAP device 2, from the result of learning based on data for six months until the therapy with the CPAP device 2 is stopped. The second period is not limited to six months and may be one month or three months.

The data analysis and prediction apparatus 4 therefore, by utilizing the generated neural network NN, can predict whether a subject potentially becomes a dropout from the CPAP device 2 in the future, depending on whether the data on the subject for the second period exhibits the same tendency as the subjects who have dropped out of the therapy with their CPAP devices 2.

For example, a professional can perform early and appropriate follow-ups for a subject likely to drop out of the therapy with the CPAP device 2, based on the prediction result of the data analysis and prediction apparatus 4, and can prevent the subject from dropping out of the therapy with the CPAP device 2.

When the specified subject is a subject at an early stage of the CPAP device 2, the learning part 42 may generate a neural network NN related to a tendency of a dropout from the CPAP device 2 from the result of learning based on data for a period shorter than the second period.

The period shorter than the second period is, for example, 10 days. A subject at an early stage of the CPAP device 2, that is, a subject who has just started to have therapy with the CPAP device 2, tends to drop out of the therapy with the CPAP device 2 early. In a case of a subject at an early stage of the CPAP device 2, therefore, the learning part 42 generates the neural network NN related to a tendency of a dropout from the CPAP device 2 from the result of learning based on data for a period shorter than the second period.

The data analysis and prediction apparatus 4 therefore, by utilizing the generated neural network NN, can predict whether a subject who has just started to have the therapy with the CPAP device 2 potentially becomes a dropout from the CPAP device 2 in the future, depending on whether the data on the subject exhibits the same tendency as a subject at an early stage who has dropped out from the therapy with the CPAP device 2.

For example, a professional can perform early and appropriate follow-ups for a subject who has just started to have the therapy with the CPAP device 2, based on the prediction result of the data analysis and prediction apparatus 4, and can prevent the subject at an early stage from dropping out of the therapy with the CPAP device 2.

When the use of the CPAP device 2 is resumed after non-use thereof for a first period or longer, the learning part 42 may exclude, from the data on the specified subjects, the data on the subject who has resumed.

Upon the lapse of the first period or longer after the use of the CPAP device 2 is stopped, the therapy with the CPAP device 2 may be resumed in some cases. For example, this is the case with overseas business trip for a long time, traveling, and hospitalization. In such a case, the therapy with the CPAP device 2 is temporarily stopped, and this case does not correspond to dropping out of the therapy with the CPAP device 2.

When the use of the CPAP device 2 is resumed, the learning part 42 excludes, from the data on the specified subjects, the data on the subject who has resumed, and generates the neural network NN.

Therefore, by utilizing the neural network NN generated excluding the data on the subject who has resumed the therapy with the CPAP device 2, the data analysis and prediction apparatus 4 can accurately predict whether a subject will drop out of the therapy with the CPAP device 2 in the future.

The data processor 41 may have a function of aggregating the data on subjects stored in the server 3 and generating use information for each subject.

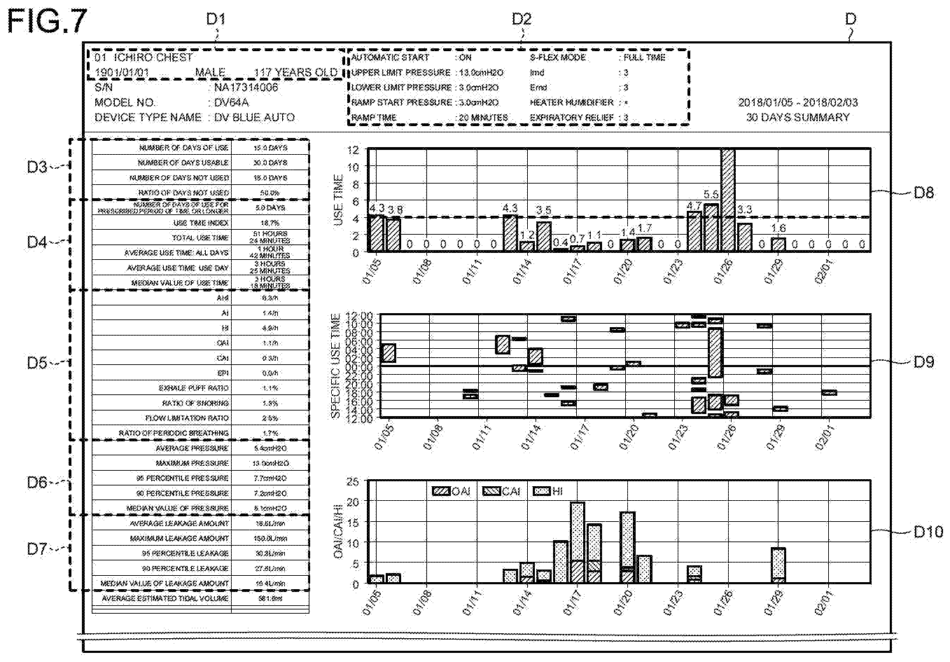

FIG. 7 is a diagram illustrating an example of use information D created for each subject by aggregating data transmitted from the CPAP devices 2. A professional can view the use information D or the printed matter of the use information D to grasp the use status of the CPAP device 2 by the subject.

The use information D includes patient attribute information D1 that is the subject's attribute information, prescription information D2 that is the setting information of the CPAP device 2, use days information D3 that is information about the number of days of use of the CPAP device 2, use time information D4 that is information about the hours of use of the CPAP device 2, apnea hypopnea information D5 that is information about apnea and hypopnea, use pressure information D6 that is information on pressure used by the CPAP device 2, leakage information D7 that is information about leakage of the CPAP device 2, a graph D8 illustrating, for example, the daily use time of the CPAP device 2 for one month, a graph D9 illustrating specific time periods in which the CPAP device 2 is used, and a graph D10 illustrating changes in OAI, CAI, and HI over time based on apnea hypopnea information D5. Elements other than those described above may be included in the use information.

The use days information D3, the use time information D4, the apnea hypopnea information D5, the use pressure information D6, and the leakage information D7 are information for grasping the use status of the CPAP device 2 for the subject. The items included in apnea hypopnea information D5 in FIG. 7 are illustrated by way of example, and other items and ratios are added with upgrading of the CPAP device 2. The learning part 42 learns from the additionally added items and ratios and generates a neural network NN related to a tendency of a dropout from the CPAP device 2 (the characteristics of the data suspicious for dropping out of the therapy with the CPAP device 2) from the result of learning.

A professional therefore can perform early and appropriate follow-ups for a subject, based on the prediction result by the analytical predictor 43 as to whether the subject will drop out of the therapy with the CPAP device 2 in the future and the use information on the use by the subject created by the data processor 41, and can prevent dropping out of the therapy with the CPAP device 2.

The learning part 42 may learn any one or more of the subject's attribute information that is the date on the specified subject, the tendency of information for a certain period about the days of use of the CPAP device 2, the tendency of information for a certain period about the use time, the tendency of information for a certain period about apnea and hypopnea, the tendency of information for a certain period about pressure, and the tendency of information for a certain period about leakage, and generate a neural network NN related to a tendency of a dropout from the CPAP device 2 from the result of learning. For example, the tendency such as whether the use of the CPAP device 2 is improving or deteriorating can be recognized, based on the tendency of information for a certain period about the days of use of the CPAP device 2.

The subject's attribute information corresponds to the patient attribute information D1, and examples thereof include the gender, the birth date, and the age. The information for a certain period about the days of use of the CPAP device 2 corresponds to the use days information D3, and examples thereof include the number of days of use for one month, the number of days usable for one month, the number of days of non-use for one month, and the ratio of days of non-use for one month. The period is not limited to one month and may be three months or six months.

The information for a certain period about the use time corresponds to the use time information D4, and examples thereof include the number of days of use of a prescribed period of time or longer in one month, the use time index in one month, the total use time in one month, the average use time in one month, and the median value of the use time in one month. The period is not limited to one month and may be three months or six months.

The information for a certain period about apnea and hypopnea corresponds to the apnea hypopnea information D5, and examples thereof include an apnea hypopnea index (AHI), an apnea index (AI), and a hypopnea index (HI).

The information for a certain period about pressure corresponds to the use pressure information D6, and examples thereof include average pressure of the CPAP device 2 for one month and the maximum pressure of the CPAP device 2 for one month. The period is not limited to one month and may be three months or six months.

The information for a certain period about leakage corresponds to the leakage information D7, and examples thereof include the average leakage amount of the CPAP device 2 for one month and the maximum leakage amount of the CPAP device 2 for one month. The period is not limited to one month, and may be three months or six months.

The learning part 42 learns based on the subject's attribute information and the like, and generates a neural network NN related to a tendency of a dropout from the CPAP device 2 from the result of learning.

The data analysis and prediction apparatus 4 therefore can predict, by utilizing the generated neural network NN, whether a subject will drop out of the therapy with the CPAP device 2 in the future, based on specific data on the subject.

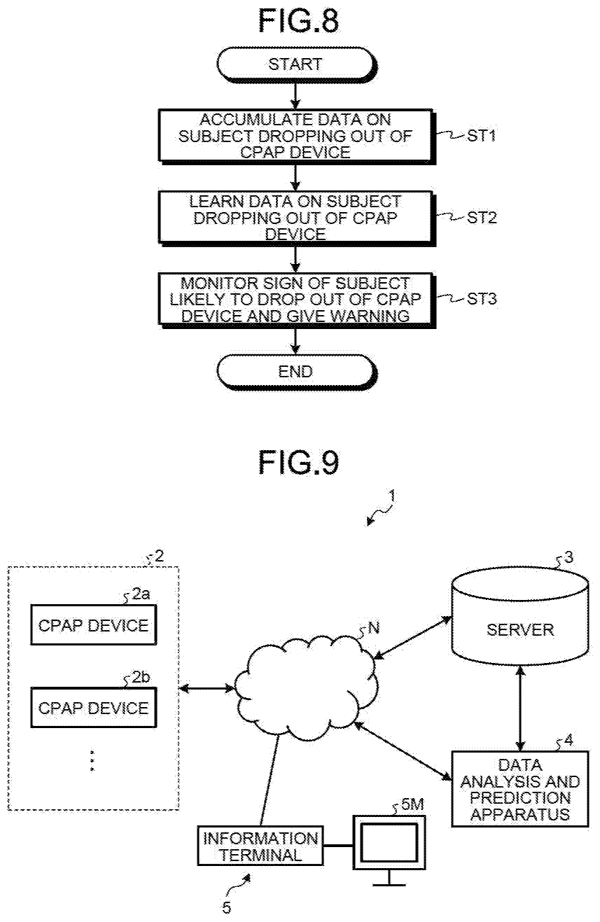

The procedure of monitoring a sign of dropping out of the CPAP device 2 performed by the data analysis and prediction apparatus 4 will now be described with reference to the flowchart in FIG. 8.

At step ST1, the data analysis and prediction apparatus 4 accumulates the data on subjects dropping out from their CPAP devices 2. For example, a subject of data (for example, the data illustrated in FIGS. 5 and 6) suspicious for dropping out of the therapy with the CPAP device 2 is specified, and the past six months of the information (for example, a variety of information illustrated in FIG. 7) on the specified subject is accumulated. A subject in the initial use of the CPAP device 2 may drop out, in which case there is no accumulation of six months of data. Thus, data for a period shorter than six months may be accumulated.

At step ST2, the data analysis and prediction apparatus 4 learns based on the data on the subject dropping out of the CPAP devices 2. Trends in six months of a variety of information are determined from the data accumulated in the process at step ST1, and a tendency of the subject dropping out of the CPAP device 2 is patterned. Examples of the items in the analysis and patterning in the process at step ST2 include the patient attribute information D1, the use days information D3, the use time information D4, the apnea hypopnea information D5, the use pressure information D6, and the leakage information D7 illustrated in FIG. 7.

At step ST3, the data analysis and prediction apparatus 4 monitors a sign of a subject likely to drop out from the CPAP device 2 and gives a warning. The data analysis and prediction apparatus 4 checks whether the use information on the use of the CPAP device 2 by the subject (for example, the daily use time of the CPAP device 2) fits the tendency (pattern) of subjects dropping out. If the use information exhibits the same or similar sign (use pattern), the data analysis and prediction apparatus 4 determines that there is a possibility that the subject will drop out of the therapy with the CPAP device 2 and provides a professional with information on this subject. When the subject is an initial user of the CPAP device 2, the data analysis and prediction apparatus 4 mainly checks whether the use information fits the pattern of subjects in the initial use from the start of use.

The data analysis and prediction apparatus 4 therefore can monitor a sign of a subject likely to drop out of the therapy with the CPAP device 2 and issue a warning to a professional based on the prediction result as to whether the subject potentially becomes a dropout from the CPAP device 2. An information terminal 5 is connected to the network N illustrated in FIG. 9. The information terminal 5 is a computer and includes a central processing unit (CPU), a random access memory (RAM), a read only memory (ROM), an internal storage device such as a hard disc drive (HDD), and a display device 5M. The data analysis and prediction apparatus 4 outputs prediction information on a prediction that a subject drops out of the therapy with the CPAP device 2, to the information terminal 5 through the network N. When having acquired the prediction information, the information terminal 5, for example, displays a warning on the display device 5M.

In the server 3, data on a subject predicted by the analytical predictor 43 to potentially become a dropout from the CPAP device 2 and a setting value of the CPAP device 2 to be used by the subject under an instruction by a professional are stored in association with each other.

The learning part 42 learns based on the data on the subject who is predicted by the analytical predictor 43 to potentially become a dropout based on the data on the subject and the setting value of the CPAP device 2 to be used by the subject that are stored in association with each other in the server 3 but has not dropped out by changing the setting value of the CPAP device 2, and the after-change setting value of the CPAP device 2. The learning module 42 generates a neural network NN from the result of learning.

The analytical predictor 43 utilizes the neural network NN generated by the learning part 42 to analyze the data processed by the data processor 41. If it is predicted that the subject potentially becomes a dropout from the CPAP device 2 in the future based on the analysis result, the analytical predictor 43 predicts the after-change setting value of the CPAP device 2 used by the subject.

Examples of the setting value of the CPAP device 2 include on/off of automatic start, upper limit pressure, lower limit pressure, ramp start pressure, and ramp time.

The learning part 42 learns based on the data on the subject who is predicted to potentially become a dropout but has not dropped out and the after-change setting value of the CPAP device 2 used by the subject, and generates a neural network NN from the result of learning.

The data analysis and prediction apparatus 4 therefore can predict, by utilizing the generated neural network NN, how the setting value of the CPAP device 2 is to be changed for a subject who potentially becomes a dropout from the CPAP device 2 in the future. Although inappropriate settings of the CPAP device 2 may lead to dropping out of the therapy with the CPAP device 2, the data analysis and prediction apparatus 4 can predict how the setting value of the CPAP device 2 is to be changed, thereby effectively preventing dropping out of the therapy with the CPAP device 2.

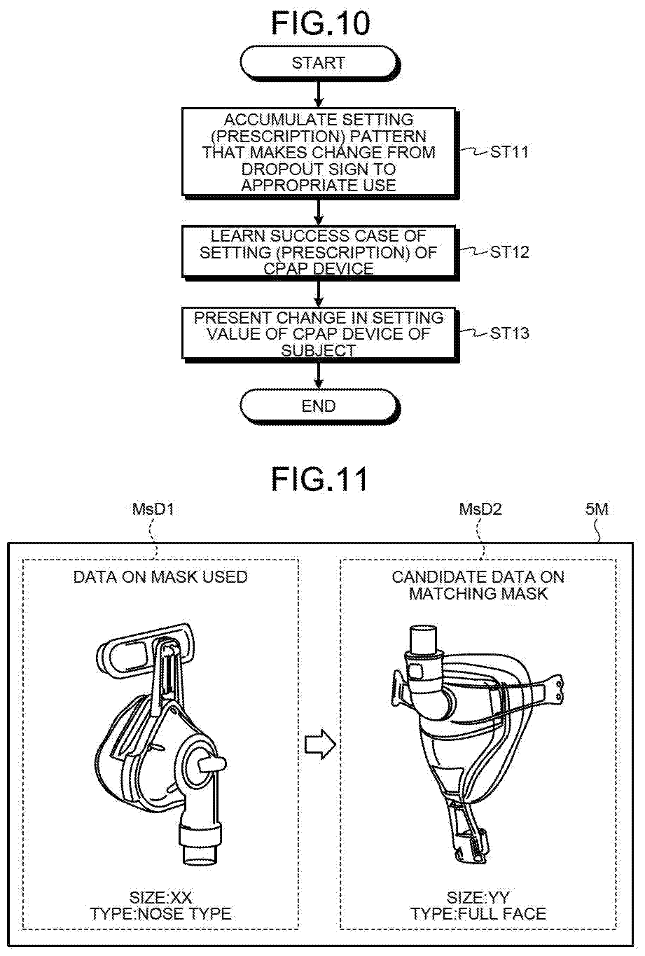

The procedure of preventing dropping out of the CPAP device 2 performed by the data analysis and prediction apparatus 4 will now be described with reference to the flowchart in FIG. 10.

At step ST11, the data analysis and prediction apparatus 4 accumulates the after-change setting (prescription) pattern of the CPAP device 2 in a case where there is a sign of dropping out of the therapy with the CPAP device 2 but changing the setting of the CPAP device 2 makes a change to an appropriate use of the CPAP device 2.

At step ST12, the data analysis and prediction apparatus 4 learns about success cases of the setting (prescription) of the CPAP device 2. The data analysis and prediction apparatus 4 determines, based on the data accumulated by the process at step ST11, the setting (prescription) that makes a change to appropriate use of the CPAP device 2, and patterns the setting (prescription) that makes a change to the appropriate use. This pattern is a success case.

The data analysis and prediction apparatus 4 holds change date information indicating when the setting of the CPAP device 2 was changed, based on the data (prescription information D2 that is setting information) transmitted from the CPAP device 2. For example, based on the change date information, the data analysis and prediction apparatus 4 inserts a mark or a sign to indicate the date of change in the graph D8 illustrating the daily use time of the CPAP device 2 or the graph D9 illustrating specific time periods during which the CPAP device 2 is used. The data analysis and prediction apparatus 4 may determine, based on data on the use state before the setting of the CPAP device 2 is changed and data on the use state after the setting of the CPAP device 2 is changed, the setting (prescription) that makes a change to the appropriate use of the CPAP device 2 and pattern the setting (prescription) that makes a change to the appropriate use.

The data analysis and prediction apparatus 4 may accumulate professional setting patterns (prescription). For example, the data analysis and prediction apparatus 4 sets a specialized medical institution and accumulates the settings made therein individually for each patient attribute information or each CPAP device 2. In this configuration, the data analysis and prediction apparatus 4 determines, based on the data accumulated by the process at step ST11 and the professional setting (prescription) pattern, the setting (prescription) that makes a change to the appropriate use of the CPAP device 2 and patterns the setting (prescription) that makes a change to the appropriate use. That is, only the setting (prescription) pattern that makes a change to the appropriate use and that matches the professional setting (prescription) pattern serves as a success case.

At step ST13, the data analysis and prediction apparatus 4 predicts and presents how the setting value of the CPAP device 2 of a subject likely to drop out of the therapy with the CPAP device 2 is to be changed.

Specifically, when it is determined that there is a possibility that the subject will drop out of the therapy with the CPAP device 2 in the process at step ST3, the data analysis and prediction apparatus 4 extracts a fitting one from among the patterned settings (prescriptions) in the process at step ST12 and presents the extracted setting (prescription).

The data analysis and prediction apparatus 4 therefore can provide a subject who potentially becomes a dropout from the CPAP device 2 in the future with how the setting value of the CPAP device 2 is to be changed.

Therapy with the CPAP device 2 uses a mask, and selecting a mask is an important factor to keep the therapy with the CPAP device 2. It is necessary to select a mask with a size that matches a subject, since the shape of nose and the length under nose vary among subjects. In addition, there are different kinds of masks, such as nose type, nostrils type, and full face type.

The data analysis and prediction apparatus 4 according to the present embodiment has the function of presenting the size and the type of a mask that matches the subject.

In the server 3, data on a subject predicted by the analytical predictor 43 to be a potential dropout from the CPAP device 2 and information about the mask to be used by the subject are stored in association with each other. The information about the mask is the size and the type of the mask.

The learning part 42 learns based on the data on the subject who is predicted by the analytical predictor 43 to be a potential dropout based on the data on the subject and the information about the mask that are stored in association with each other in the server 3 but has not dropped out by changing the mask, and information about the after-change mask, and generates a neural network NN from the result of learning.

The analytical predictor 43 utilizes the neural network NN to analyze the data processed by the data processor 41. If it is predicted that the subject potentially becomes a dropout from the CPAP device 2 in the future based on the analysis result, the analytical predictor predicts information about a mask that matches the subject.

For example, the data analysis and prediction apparatus 4 determines information about a mask when the use of the CPAP device 2 changes to an appropriate use, based on the apnea hypopnea information D5, the use pressure information D6, and the leakage information D7 before the mask is changed, and the apnea hypopnea information D5, the use pressure information D6, and the leakage information D7 after the mask is changed, and patterns information about the mask when the use of the CPAP device 2 changes to the appropriate use.

If the data analysis and prediction apparatus 4 determines that there is a possibility that the subject will drop out of the therapy with the CPAP device 2, it extracts the one that matches the subject from the patterned information about the mask, and presents the extracted information about the mask.

FIG. 11 is a diagram illustrating a display example of mask information displayed on the information terminal. The data analysis and prediction apparatus 4 outputs the extracted information about the mask to the information terminal 5 through the network N. After acquiring the extracted information about the mask, the information terminal 5, for example, displays information MsD1 on the mask that has been used and extracted information MsD2 about the matching candidate mask, for example, on the display device 5M. Supported by the information terminal 5 and the display device 5M, the professional easily selects a mask.

A professional therefore can select a mask that matches a subject, based on the prediction result by the analytical predictor 43 as to whether the subject will drop out of the therapy with the CPAP device 2 in the future and the information about the mask of the subject, and can prevent dropping out of the therapy with the CPAP device 2.

The data analysis and prediction apparatus 4 may include a changer 44 configured to change the setting value of the target CPAP device 2 based on an after-change setting value when the analytical predictor 43 predicts an after-change setting value of the CPAP device 2 used by the subject.

For example, the changer 44 specifies the target CPAP device 2 from the data transmitted from the CPAP device 2 (the number (S/N) unique to the CPAP device 2 and/or the media access control (MAC) address of the CPAP device 2) and accesses the specified CPAP device 2 through the communicator 40 to change the setting value of the CPAP device 2. The setting value of the CPAP device 2 may be changed by the server 3. In this configuration, the changer 44 accesses the server 3 through the communicator 40 and provides information (for example, MAC address) on the specified CPAP device 2 and the setting value to be changed. The server 3 accesses the CPAP device 2 specified by the changer 44 and changes the setting value of the CPAP device 2.

For example, the data processor 4 extracts, for example, information about apnea and hypopnea and information about use time from the data on a subject transmitted from the CPAP device 2. The analytical predictor 43 provides the information about apnea and hypopnea and the information about use time obtained by the data processor 4 to the neural network NN of the learning part 42 and acquires the setting value of the CPAP device 2. The changer 44 accesses the specified CPAP device 2 through the communicator 40 and changes the setting value of the CPAP device 2. For example, the setting value of the CPAP device 2 is changed so as to reduce the setting pressure at the onset of sleep and to increase the setting pressure after the onset of sleep from the current setting.

With such a configuration, the data analysis and prediction apparatus 4 can change the setting value of the CPAP device 2 of a subject who potentially becomes a dropout from the CPAP device 2 in the future, to a suitable setting value, and can prevent dropping out of the therapy with the CPAP device 2.

In the present embodiment, the configuration and operation of the data analysis and prediction apparatus 4 for predicting whether a subject potentially becomes a dropout from the CPAP device 2 in the future has been described. However, the present embodiment is not limited thereto and may be configured as a data analysis and prediction program including the components for predicting whether a subject potentially becomes a dropout from the CPAP device 2 in the future.

The data analysis and prediction program may be recorded on a computer-readable recording medium, and the data analysis and prediction program recorded on the recording medium may be read and executed by a computer to implement such a configuration.

Specifically, the data analysis and prediction program is a computer program causing a computer to execute: a data processing step of processing data on a subject transmitted from a CPAP device 2; a learning step of learning based on data on a dropout who drops out of therapy with the CPAP device 2, based on data on subjects stored in a server, which stores therein data on a plurality of subjects transmitted from a plurality of CPAP devices 2, and generating a neural network NN from the result of learning; and an analysis and prediction step of analyzing the data processed by the data processing step using the neural network NN generated by the learning step, and predicting whether a subject potentially becomes a dropout from the CPAP device 2 in the future, based on the analysis result.

### Second Embodiment

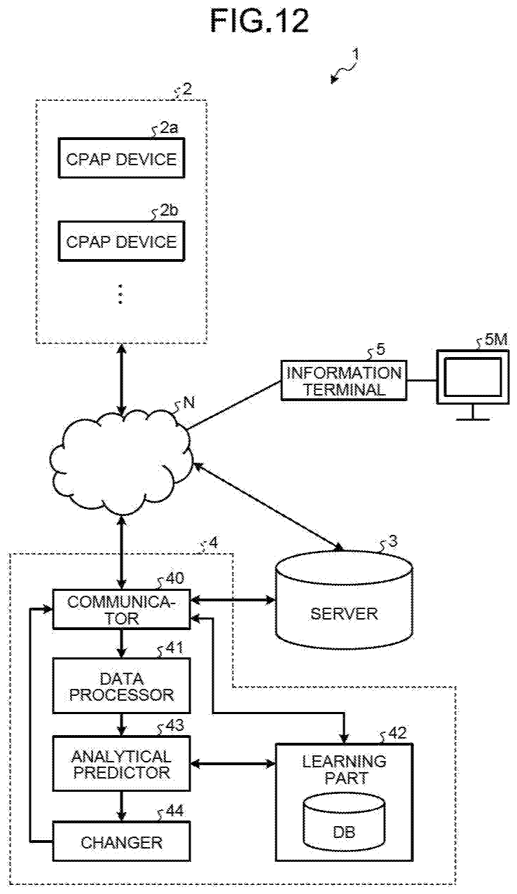

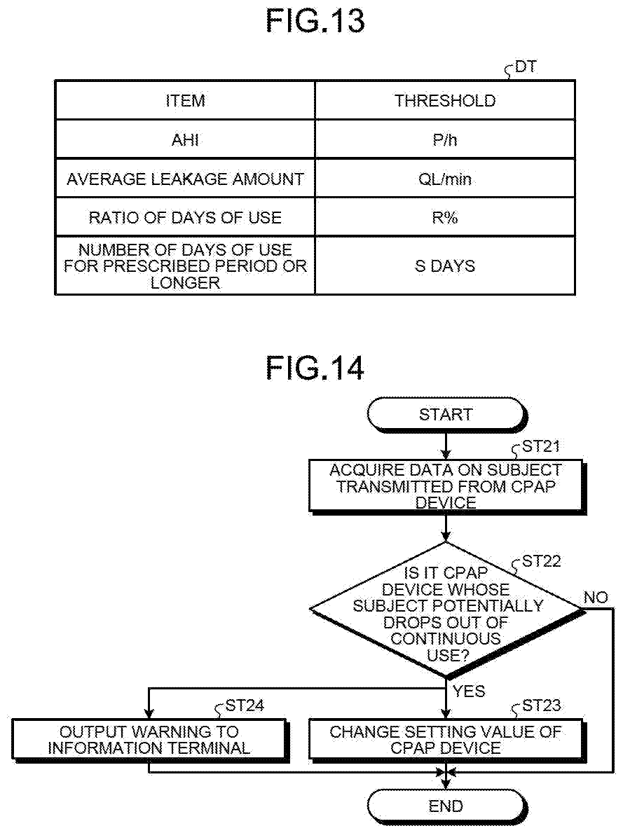

FIG. 12 is a diagram illustrating a configuration of the CPAP management system in a second embodiment. FIG. 13 is a diagram illustrating a database for determining a sign of dropping out of the CPAP device. The same components as illustrated in the foregoing embodiment are denoted by the same reference signs and an overlapping description will be omitted. FIG. 14 is a flowchart illustrating the procedure of monitoring a sign of dropping out of the CPAP device.

In the second embodiment, the learning part 42 includes a database DB in a storage device. The database DB has reference data DT serving as criteria for determining whether a subject potentially becomes a dropout from therapy with the CPAP device 2, illustrated in FIG. 13. For example, the items of the reference data DT include the above-mentioned AHI, the average leakage amount, the ratio of the days of use thereof, and the days of use for a prescribed period of time or longer.

Data on a plurality of subjects transmitted from a plurality of CPAP devices 2 are stored in the server 3. Thresholds P, Q, R, and S of the reference data DT are set based on the data on subjects stored in the server 3. Specifically, the thresholds P, Q, R, and S of the reference data DT are set by the learning part 42 by extracting the average of data for the second period until the date on which the use of the CPAP devices 2 are stopped, from among data on a plurality of subjects whose periods of non-use of the CPAP devices 2 are equal to or longer than the first period included in data on a plurality of subjects.

As illustrated in FIG. 14, the data analysis and prediction apparatus 4 acquires the data on a subject transmitted from the CPAP device 2 through the network N and accumulated in the server 3 (step ST21).

Subsequently, the data processor 41 processes the data on the subject and computes the respective average values of the measurement value of AHI, the average leakage amount, the ratio of the days of use thereof, and the days of use for a prescribed period of time or longer.

Subsequently, the analytical predictor 43 provides the respective average values of the measurement value of AHI, the average leakage amount, the ratio of the days of use thereof, and the days of use for a prescribed period of time or longer to the database DB of the learning part 42 and conducts analysis for the analysis target CPAP device 2. Specifically, when the average value of the measurement value of the AHI is greater than the threshold P/h, the average leakage amount is greater than the threshold QL/min, the average of the ratio of the days of use thereof is smaller than the threshold R%, and the average value of the days of use for a prescribed period of time or longer is smaller than the threshold S days, it is determined that the analysis target CPAP device 2 is a CPAP device whose subject will potentially drop out of continuous use. If it is determined that the analysis target CPAP device 2 is a CPAP device whose subject will potentially drop out of continuous use (Yes at step ST22), the data analysis and prediction apparatus 4 proceeds to step ST23 and step ST24. If it is determined that the analysis target CPAP device 2 is a CPAP device whose subject will potentially drop out of continuous use (step ST22, No), the data analysis and prediction apparatus 4 terminates the process.

The changer 44 specifies the analysis target CPAP device 2 from the data transmitted from the CPAP device 2 (the number (S/N) unique to the CPAP device 2 and/or the MAC address of the CPAP device 2) and accesses the specified CPAP device 2 through the communicator 40 to change the setting value of the CPAP device 2 (step ST23).

The data analysis and prediction apparatus 4 outputs prediction information that the subject will drop out of the therapy with the CPAP device 2 to the information terminal 5 through the network N. When having acquired the prediction information, the information terminal 5, for example, displays a warning on the display device 5M (step ST24).

Although preferred embodiments have been described above, the present disclosure is not limited to such embodiments. The contents disclosed in the embodiments are illustrated only by way of example and are susceptible to various modifications without departing from the spirit of the present disclosure. The modifications made as appropriate without departing from the spirit of the present disclosure should belong to the technical scope of the present disclosure.

The present embodiment also includes the following aspects. A data analysis and prediction apparatus in an aspect includes: a data processor configured to process data on a subject transmitted from a therapeutic device; a learning part configured to learn based on data on a dropout who drops out of therapy with the therapeutic device, based on data on a plurality of subjects stored in a server, which stores data on the subjects transmitted from a plurality of therapeutic devices, and generate a neural network from the result of learning; and an analytical predictor configured to analyze the data processed by the data processor using the neural network and predict whether a subject potentially becomes a dropout from the therapeutic device in the future, based on the analysis result.

With this configuration, the data analysis and prediction apparatus can predict, by utilizing artificial intelligence (AI), whether a subject will drop out of therapy with a therapeutic device (for example, CPAP device) in the future. For example, the result of prediction by the data analysis and prediction apparatus is presented to a professional, so that the professional can perform early and appropriate follow-ups for the subject and prevent the subject from dropping out of the therapy with the therapeutic device. Examples of the professional include health workers such as doctors, laboratory technicians, and nurses.

The learning part specifies data on a subject whose period of non-use of the therapeutic device is a first period or longer, based on the data on a plurality of subjects, and generates the neural network related to a tendency of a dropout from the therapeutic device from the result of learning based on the data on the specified subject.

The first period is, for example, 14 days. Upon the lapse of the first period or longer after the use of the therapeutic device is stopped, dropping out of the therapy with the therapeutic device is suspected. The data analysis and prediction apparatus learns based on data on a dropout from the therapeutic device, based on data on a subject who has dropped out of the therapy with the therapeutic device, and generates a neural network from the result of learning. The data analysis and prediction apparatus therefore can predict, by utilizing the generated neural network, whether a subject potentially becomes a dropout from the therapeutic device in the future, depending on whether data on the subject exhibits the same tendency as a subject dropping out of the therapy with the therapeutic device. For example, a professional can perform early and appropriate follow-ups for a subject likely to drop out of the therapy with the therapeutic device and can prevent the subject from dropping out of the therapy with the therapeutic device.

The learning part extracts data for a second period from the data on the specified subject, learns based on the extracted data for the second period, and generates the neural network related to a tendency of a dropout from the therapeutic device from the result of learning.

The second period is, for example, six months until the first day on which the use of the therapeutic device is stopped. The second period is not limited to six months and may be one month or three months. The learning part learns based on data for six months before the therapy with the therapeutic device is stopped and generates a neural network from the result of learning. The data analysis and prediction apparatus therefore, by utilizing the generated neural network, can predict whether a subject potentially becomes a dropout from the therapeutic device in the future, depending on whether data on the subject for the second period exhibits the same tendency as a subject dropping out of the therapy with the therapeutic device. For example, a professional can perform early and appropriate follow-ups for a subject likely to drop out of the therapy with the therapeutic device and can prevent the subject from dropping out of the therapy with the therapeutic device.

When the specified subject is a subject at an early stage of the therapeutic device, the learning part may learn based on data for a period shorter than the second period and generate the neural network related to a tendency of a dropout from the therapeutic device from the result of learning.

The period shorter than the second period is, for example, 10 days. A subject at an early stage of the therapeutic device, that is a subject who has just started to receive the therapy with the therapeutic device, tends to drop out of the therapy with the therapeutic device early. In a case of a subject at an early stage of the therapeutic device, therefore, the learning part learns based on data for a period shorter than the second period and generates a neural network from the result of learning. The data analysis and prediction apparatus therefore can predict, by utilizing the generated neural network, whether a subject who has just started to receive the therapy with the therapeutic device potentially becomes a dropout from the therapeutic device in the future, depending on whether the data on the subject exhibits the same tendency as a subject at an early stage who has dropped out of the therapy with the therapeutic device. For example, a professional can perform early and appropriate follow-ups for a subject who has just started to receive the therapy with the therapeutic device and can prevent the subject at an early stage from dropping out of the therapy with the therapeutic device.

When the use of the therapeutic device is stopped for the first period or longer, but the use of the therapeutic device is resumed later, the learning part may exclude, from the data on the specified subjects, data on the subject who has resumed.

Upon the lapse of the first period or longer after the use of the therapeutic device is stopped, the therapy with the therapeutic device may be resumed in some cases. For example, this is the case with overseas business trip for a long time, traveling, and hospitalization. In such a case, the therapy with the therapeutic device is temporarily stopped, and this case does not correspond to dropping out of the therapy with the therapeutic device. When the use of the therapeutic device is resumed, the learning part excludes data on the subject who has resumed from the data on the specified subjects, and generates a neural network. The data analysis and prediction apparatus therefore can accurately predict whether the subject will drop out of the therapy with the therapeutic device in the future, by utilizing the neural network generated excluding the data on the subject who has resumed the therapy with the therapeutic device.

The learning part may learn one information or a plurality of information of subject's attribute information that is the data on the specified subject, information about the days of use of the therapeutic device, information about the use time, information about apnea and hypopnea, information about pressure, and information about leakage, and generate the neural network related to a tendency of a dropout from the therapeutic device from the result of learning.

The subject's attribute information is, for example, the gender, the birth date, and the age. The information about the days of use of the therapeutic device is, for example, the number of days of use for one month, the number of days usable for one month, the number of days of non-use for one month, and the ratio of the days of non-use for one month. The information about the use time is, for example, the days of use for a prescribed period of time or longer in one month, the ratio of the days of use for a prescribed period of time or longer in one month, the total use time in one month, the average use time in one month, and the median value of the use time in one month. The information about apnea and hypopnea is, for example, an apnea hypopnea index (AHI), an apnea index (AI), and a hypopnea index (HI). The information about pressure is, for example, the average pressure of the therapeutic device for one month and the maximum pressure of the therapeutic device for one month. The information about leakage is, for example, the average leakage amount of the therapeutic device for one month and the maximum leakage amount of the therapeutic device for one month. The learning part learns based on data on a dropout from the therapeutic device from the subject's attribute information and the like, and generates a neural network from the result of learning. The data analysis and prediction apparatus therefore can predict, by utilizing the generated neural network, whether a subject will drop out from the therapy with the therapeutic device in the future, based on the specific data on the subject.

In the server, data on a subject predicted by the analytical predictor to potentially become a dropout from the therapeutic and a setting value of the therapeutic device to be used by the subject under an instruction by a professional are stored in association with each other. The learning part may learn based on the data on the subject who is predicted by the analytical predictor to potentially become a dropout, based on the data on the subject and the setting value of the therapeutic device to be used by the subject that are stored in association with each other in the server, but has not dropped out by changing the setting value of the therapeutic device, and the after-change setting value of the therapeutic device, and may generate a neural network from the result of learning. The analytical predictor may utilize the neural network to analyze the data processed by the data processor and, if it is predicted that the subject potentially becomes a dropout from the therapeutic device in the future based on the analysis result, predict an after-change setting value of the therapeutic device used by the subject.

Examples of the setting value of the therapeutic device include on/off of automatic start, upper limit pressure, lower limit pressure, ramp start pressure, and ramp time. Inappropriate settings of the therapeutic device may lead to dropping out of the therapy with the therapeutic device. The learning part learns based on the data on the subject who is predicted to potentially become a dropout but has not dropped out and the after-change setting value of the therapeutic device of the subject, and generates a neural network from the result of learning. The data analysis and prediction apparatus therefore can predict, by utilizing the generated neural network, how the setting value of the therapeutic device is to be changed for a subject who potentially becomes a dropout from the therapeutic device in the future.

The data analysis and prediction apparatus may include a changer configured to change the setting value of the target therapeutic device, based on an after-change setting value, when the analytical predictor predicts an after-change setting value of the therapeutic device used by the subject.

The data analysis and prediction apparatus can change the setting value of the therapeutic device of a subject who potentially becomes a dropout in the future, to a suitable setting value, and can prevent dropping out of the therapy with the therapeutic device.

In the server, the data on a subject predicted by the analytical predictor to potentially become a dropout from the therapeutic device and information about a mask to be used by the subject are stored in association with each other. The learning part may learn based on the data on the subject who is predicted by the analytical predictor to potentially become a dropout, based on the data on the subject and the information about the mask that are stored in association with each other in the server, but has not dropped out by changing the mask, and information on the changed mask, and may generate a neural network from the result of learning. The analytical predictor may utilize the neural network to analyze the data processed by the data processor and, if it is predicted that the subject potentially becomes a dropout from the therapeutic device in the future based on the analysis result, predict information on a mask suitable for the subject.

A professional such as a doctor can select a mask that matches the subject, based on the prediction result by the analytical predictor as to whether the subject will drop out of the therapy with the therapeutic device in the future and the information about the mask of the subject, and can prevent dropping out of the therapy with the therapeutic device.

A data analysis and prediction program according to the present embodiment is a computer program causing a computer to execute: a data processing step of processing data on a subject transmitted from a therapeutic device; a learning step of learning based on data on a dropout who drops out of the therapy with the therapeutic device, based on data on subjects stored in a server, the server storing data on a plurality of subjects transmitted from a plurality of therapeutic devices, and generating a neural network from the result of learning; and an analysis and prediction step of analyzing the data processed by the data processing step using the neural network and predicting whether a subject potentially becomes a dropout from the therapeutic device in the future, based on the analysis result.

With this configuration, the data analysis and prediction program can predict, by utilizing artificial intelligence (AI), whether a subject will drop out of the therapy with the therapeutic device in the future. For example, the result of prediction by the data analysis and prediction program is presented to a professional, so that the professional can perform early and appropriate follow-ups for the subject and prevent the subject from dropping out of therapy using the therapeutic device.

For other operation effects brought about by the aspects described in the present embodiment, those apparent from the present disclosure or those conceivable by a person skilled in the art should be understood to be brought about by these aspects.

### REFERENCE SIGNS LIST

1 CPAP management system

2 CPAP device

3 server

4 data analysis and prediction apparatus

40 communicator

41 data processor

42 learning part

43 analytical predictor

44 changer

1. A CPAP management system comprising:
a data processor configured to process data on a subject transmitted from a CPAP device; and
an analytical predictor configured to
extract data for a second period until a date on which use of the CPAP device is stopped, from data on a subject whose period of non-use of the CPAP device is a first period or longer included in data on a plurality of subjects stored in a server that stores therein the data on the subjects transmitted from a plurality of the CPAP devices, and
output a prediction result as to whether the subject potentially becomes a dropout from the CPAP device in the future, based on the data for the second period.

2. The CPAP management system according to claim 1, further comprising
a learning part configured to
learn based on data on a dropout who drops out of therapy with the CPAP device and
generate a neural network from a result of learning, wherein
the analytical predictor utilizes the neural network to analyze data processed by the data processor.

3. The CPAP management system according to claim 1, further comprising a setting changer configured to output a command to change a setting of the CPAP device in accordance with the prediction result.

4. The CPAP management system according to claim 1, wherein, for the CPAP device from which the subject potentially drops out, information that matches the subject is extracted from among patterned information about a mask, and the extracted information about another mask is presented to an information terminal.

5. A management method of managing a plurality of CPAP devices, the management method comprising:
a first step of
storing data on a plurality of subjects transmitted from the CPAP devices into a server,
extracting, from data on a subject whose period of non-use of a corresponding CPAP device of the CPAP devices is equal to or longer than a first period included in the data on subjects stored in the server, data for a second period until a date on which use of the CPAP device is stopped, and
creating reference data about a tendency of a dropout from the CPAP devices;
a second step of pre-processing data on a subject transmitted from a CPAP device of the CPAP devices; and
a third step of
analyzing the data on the subject acquired in the second step based on the reference data created in the first step and
outputting a warning that the subject potentially drops out of therapy with the CPAP device.

6. The CPAP management system according to claim 2, further comprising a setting changer configured to output a command to change a setting of the CPAP device in accordance with the prediction result.
